(19) United States
(12) Patent Application Publication
(10) Pub. No.: US 2008/0228257 A1
(43) Pub. Date: Sep. 18, 2008

(54) Covered stent balloon and method of using same

(76) Inventor: Jacob Richter, Ramat Hasharon (IL)

Correspondence Address: MORGAN & FINNEGAN, L.L.P., 3 World Financial Center, New York, NY, 10281-2101, US

(21) Appl. No.: 11/725,072

(22) Filed: Mar. 15, 2007

(51) Int. Cl. A61F 2/06

(52) U.S. Cl. 623/1.11

(57) ABSTRACT

An improved stent delivery system which provides a covered stent as a section of a deployment balloon is disclosed. The invention satisfies the need for a highly deliverable stent system with maximum flexibility, reduced profile, and consistent, smooth shaping along its length. The stent system described further minimizes damage to a drug-eluting coating and promotes uniform expansion of the stent during deployment. The invention provides a stent delivery system wherein a stent has a substantially impermeable cover in which the cover is detachably associated with a catheter shaft. The cover and the stent detach from the catheter shaft upon expansion of the stent to a desired diameter. In one embodiment, detachment is achieved automatically. In another embodiment, detachment is manual.

### FIELD OF THE INVENTION

The present invention relates generally to stents, which are endoprostheses implanted into vessels within a living body, such as blood vessels, to support and hold open the vessels. More particularly, the present invention relates to improvements in stent delivery systems using a covered stent as part of, or integral with, a catheter balloon.

### BACKGROUND OF THE INVENTION

Various stents are known in the art. Generally, stents are used to treat stenosis—the narrowing or constriction of a body vessel. Over time, fat, cholesterol, and other substances in the bloodstream can accumulate on the vessel walls, restricting blood flow. At the location of constriction, a stent can be implanted and expanded, opening the constriction and restoring blood flow. Stents are generally tubular in shape, and are expandable from a relatively small, unexpanded diameter to a larger, expanded diameter. Stents are typically implanted with a catheter. In the case of a balloon-expandable stent, the stent is crimped onto a balloon catheter. The catheter is then maneuvered through the patient's vasculature to the desired deployment location—in the area of the constriction. By inflating the balloon, the stent is expanded and effectively secured in place against the vessel wall. The expanded stent remains in the body vessel to provide support to the vessel, minimize the effect of the original constriction, and improve blood flow. However, stented vessels are susceptible to restenosis, or the re-narrowing of the widened vessel. Restenosis can occur over the length of the stent and/or past the ends of the stent. As a means of reducing restenosis, conventional stents have been coated with drugs which inhibit smooth muscle cell proliferation.

Drug coated stents are well known in the art. One approach involves applying to the stent prior to insertion a thin polymer film that is loaded with an inhibiting drug. The release characteristics of the drug generally depend on the nature of the polymeric material as well as the drug to be incorporated. The drug may diffuse out of the polymer or be released through degradation of the polymeric material. Thus, the release rate of the drug from the coating is an important consideration. Moreover, it is important to avoid damage to the drug coating during implantation of the stent because such damage can adversely affect the release rate as well as reduce the available supply of the drug.

Balloon catheters are a well-known means for intraluminal delivery and implant of a stent. It is to be understood that in conventional stent delivery systems, the balloon and stent are separate, distinct components. After the stent is deployed, the balloon is removed from the body leaving the stent implanted. A drawback of conventional balloon catheters is their lack of flexibility. Typically, a balloon catheter is relatively stiff across the portion on which the stent is crimped, which makes delivering the stent through a tortuous vessel difficult. Another drawback of a catheter's relative inflexibility is its tendency to straighten arched blood vessels, which may damage the blood vessel and create undesirable blood circulation problems.

Another shortcoming of conventional balloon catheters is that they may cause the ends of the stent to flare out during implantation. This problem sometimes manifests itself in a phenomenon called “dog-boning”. The projecting edges can increase trauma to the wall of the vessel during stent deployment.

In some instances, it may be advantageous to use a stent that has been covered with a biocompatible material. A cover can be used to reduce adverse tissue reactions associated with contact between the expanded stent and the walls of the body vessel. Covered stents also help prevent the struts of the stent from cutting into the plaque of the stenosis, reducing the possibility of forming embolic debris that can be released into the blood stream. Another benefit is that, during expansion of the stent, the covering can trap embolic particles against the arterial wall, preventing their release into the blood stream. A cover can also be used to control the expansion characteristics of a stent as it is deployed with a balloon. Depending on the nature of the covering, other advantages will be realized. For example, if a biodegradable covering is used, less physical material will ultimately remain in the vessel over the long term.

Despite the potential advantages, the combination of a covered stent and balloon catheter can nonetheless suffer certain disadvantages. For example, while a stent may be sufficiently flexible prior to being mounted on a balloon, the flexibility of the combination is limited by the balloon's characteristics. This can adversely affect the system's deliverability, or ability to be manipulated through tortuous vessels. Crimping a covered stent on a balloon increases the system's profile, which also adversely affects deliverability. Furthermore, if a drug eluting covered stent is utilized, interaction with the balloon can damage the drug coating, potentially reducing the efficacy of drug delivery.

Thus, it is desired to have a highly deliverable stent system with maximum flexibility, decreased profile, and smoothness along its length, that minimizes damage to a drug-eluting coating, and promotes the uniform expansion of the deployed stent.

One object of the present invention is to provide a stent delivery system that possesses a high degree of flexibility and deliverability.

Another object of the present invention is to provide a stent delivery system with a reduced and smooth profile.

Another object of the present invention is to diminish the tendency of a stent to “dog bone” during expansion.

Yet another object of the present invention is to eliminate damage to the coating of a drug-eluting stent caused by an expanding balloon.

Still another object of the present invention is to maintain the desired positioning of the stent as it is deployed within a living body.

### SUMMARY OF THE PRESENT INVENTION

The present invention provides an improved delivery system for deploying a covered stent within tubular organs, blood vessels, or other tubular body lumens. The delivery system of the present invention provides a covered stent which itself is part of, or integral with, a deployment balloon. A sealing and a detachment system for the covered stent are provided to facilitate a smooth and low profile stent delivery system.

When the covered stent body is ready for insertion, each end of the covered stent body is sealed and attached to the catheter shaft such that the covered stent body itself will expand upon introduction of fluid through an inflation lumen of the catheter.

In one embodiment, the ends of the covered stent body are attached to sleeves which are in turn attached to the catheter shaft. The attachment between the covered stent body and the sleeves is broken when the sleeves are automatically detached at a desired stent diameter and fluid pressure. In another embodiment, the stent body covering is integral with the catheter such that there are separation lines, such as, for example, perforations, scoring or a frangible web, built into the covering at the desired detachment points. When the stent body is substantially fully expanded a separation wire is pulled by the operator. When the wire is pulled, each end of the cover is detached from the covered stent body at the separation lines. It will also be understood that the separation lines formed in the covering at the desired attachment points can be constructed to allow for automatic detachment when the stent body is substantially fully expanded to its desired diameter, eliminating the need for a separation wire.

The present invention also provides a method for deployment of a covered stent body within tubular organs, blood vessels, or other tubular body lumens. In one embodiment of the invention the method includes the steps of providing a stent body comprising a stent covered with a substantially impermeable cover integrated with a deployment catheter, the cover characterized as being conformable to all variations in stent diameter; inserting the stent body and deployment catheter system into the living body to a desired location within a vessel; expanding the covered stent body; and removing the deployment catheter. In one embodiment, the ends of the covered stent body are attached to sleeves, which are attached to the deployment catheter, such that the sleeves automatically detach from the stent body ends at a desired stent diameter and balloon pressure. In another embodiment, the stent cover is integrated with the deployment catheter at separation lines such that after substantially full expansion of the covered stent body, each end of the stent cover is separated from the deployment catheter by the pull of a separation wire, or as may occur automatically, by a separating action, such as, for example, tearing or ripping, along the separation lines created by the balloon's expansive pressure.

### BRIEF DESCRIPTION OF THE DRAWINGS

FIG. 1 shows a cross-sectional view of an embodiment of a covered stent in accordance with the present invention, taken along the longitudinal axis of the covered stent.

FIG. 2 shows a cross-sectional view of an embodiment of a stent delivery system in accordance with the present invention, similarly taken along its longitudinal axis.

FIG. 3 shows a cross-sectional view of another embodiment of a stent delivery system in accordance with the present invention, taken along its longitudinal axis.

### DETAILED DESCRIPTION OF THE INVENTION

The present invention satisfies the need for a highly deliverable stent system with maximum flexibility, reduced profile, and consistent, smooth shaping along its length. The present invention further minimizes damage to a drug-eluting coating and promotes uniform expansion of the stent during deployment.

The delivery system of the present invention comprises a generally tubular stent body made of a material capable of radial expansion. Persons of ordinary skill in the art will appreciate that the basic geometry of the stent body may take any suitable configuration such as is known for stents, and that the stent body may be formed of any suitable material, such as stainless steel, cobalt chromium, nitinol, magnesium, or any alloy meeting at least the physical property characteristics that these materials exhibit. Examples of suitable structural configurations for the stent body include, but are not limited to, those shown in U.S. Pat. No. 5,733,303 to Israel et al., or those forming part of the NIR™ family of stents manufactured by Medinol Ltd. The disclosure of U.S. Pat. No. 5,733,303 is hereby expressly incorporated by reference into this application. Other examples of suitable structural configurations include but are not limited to, those shown in U.S. Pat. Nos. 6,723,119 and 6,709,453 to Pinchasik et al., or those forming part of the NIRflex™ family of stents, also manufactured by Medinol Ltd. The disclosures of U.S. Pat. Nos. 6,723,119 and 6,709,453 are also expressly incorporated by reference into this application. Other suitable stent structures may be used in the present invention and their identification is readily known to the skilled artisan based upon the teaching of the present invention. Depending upon the desired application, the stent may comprise a bare stent or may have a drug-eluting coating, such as, for example, that described in U.S. Pat. No. 7,163,555, the disclosure of which is incorporated herein by reference.

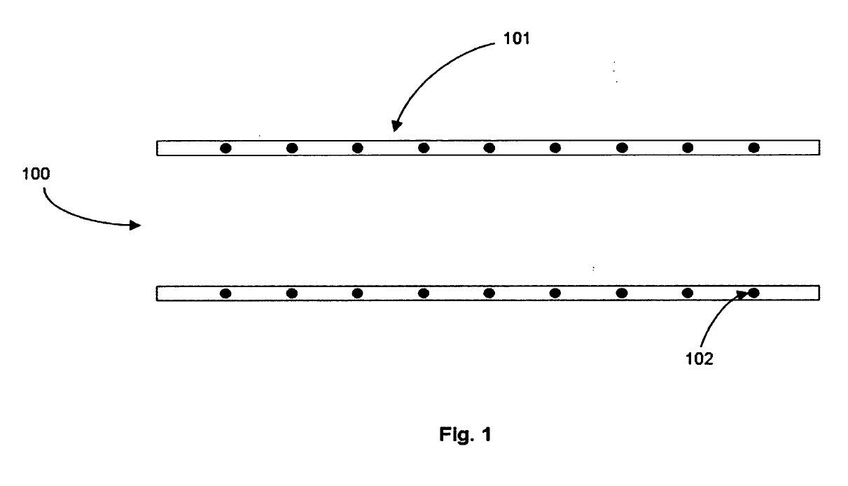

An illustrative embodiment of a covered stent in accordance with the present invention is schematically represented in FIG. 1, wherein a stent 100 is covered with a stent cover 101, which is a biocompatible barrier material. Stent cover 101 may be wrapped around the abluminal surface of stent 100, or as shown, struts 102 of stent 100 may be embedded within stent cover 101. Stent cover 101 may be constructed of suitable fluoropolymer materials such as polytetrafluoroethylene (PTFE) or copolymers of tetrafluoroethylene with other monomers. Such monomers include ethylene, chlorotrifluoroethylene, perfluoroalkoxytetrafluoroethylene, or fluorinated propylenes such as hexafluoropropylene. Expanded PTFE (ePTFE) may also be used. Examples of suitable cover materials are described in U.S. Pat. Nos. 5,749,880, 6,808,533 and in published U.S. Pat. Application No. 2006/0271165, the disclosures of which are expressly incorporated herein. Stent cover 101 will be understood to be capable of conforming to variations in the shape and diameter of stent 100. Depending upon the desired application, stent cover 101 may be a drug-eluting stent cover which may either be coated with a drug or constructed such that the drug is embedded within the stent cover.

Stent 100 and/or stent cover 101 may also be biodegradable or bioresorbable. A bioresorbable or biodegradable material is a material that is absorbed or is degraded in the body by active or passive processes. When either type of material is referred to in the foregoing description, it is meant to apply to both bioresorbable and biodegradable materials. Suitable materials are described in published U.S. Pat. Application No. 2005/0033399 and U.S. Pat. No. 6,939,376, both of which are incorporated herein by reference in there entirety. Some examples include polyesters, polyanhydrides, polyglycolide and trimethylene carbonate.

There are several advantages of using the bioresorbable material. It does not obscure radiographs or MRI/CT scans, which allows for more accurate evaluation during the healing process. Another advantage of using the bioresorbable material is that the continuous covering provided by the bioresorbable material after the stent is deployed in a vessel is believed to inhibit or decrease the risk of embolization. Another advantage is the prevention of “stent jail” phenomenon, or the complication of tracking into side branches covered by the stent.

The depletion of the bioresorbable material covering can be controlled by modification or choosing characteristics of the bioresorbable material to allow degradation at a time about when the stent body is fixated in the vessel wall and embolization is no longer a risk.

The bioresorbable material can be any material that readily degrades in the body and can be naturally metabolized. For example, the bioresorbable material can be, but is not limited to, a bioresorbable polymer. If selected, any bioresorbable polymer can be used with the present invention, such as polyesters, polyanhydrides, polyorthoesters, polyphosphazenes, and any of their combinations in blends or as copolymers. Other usable bioresorbable polymers can include polyglycolide, polylactide, polycaprolactone, polydioxanone, poly(lactide-co-glycolide), polyhydroxybutyrate, polyhydroxyvalerate, trimethylene carbonate, and any blends and copolymers of the above polymers.

Synthetic condensation polymers, as compared to addition type polymers, are generally biodegradable to different extents depending on chain coupling. For example, the following types of polymers biodegrade to different extents (polyesters biodegrade to a greater extent than polyethers, polyethers biodegrade to a greater extent than polyamides, and polyamides biodegrade to a greater extent than polyurethanes). Morphology is also an important consideration for biodegradation. Amorphous polymers biodegrade better than crystalline polymers. Molecular weight of the polymer is also important. Generally, lower molecular weight polymers biodegrade better than higher molecular weight polymers. Also, hydrophilic polymers biodegrade faster than hydrophobic polymers. There are several different types of degradation that can occur in the environment. These include, but are not limited to, biodegradation, photodegradation, oxidation, and hydrolysis. Often, these terms are combined together and called biodegradation. However, chemists and biologists generally consider these processes, i.e., biodegradation, photodegradation and oxidation, to be separate and distinct. Biodegradation alone involves enzymatically-promoted breakdown of the polymer caused by living organisms.

As a further advantage of the invention, the bioresorbable material may be embedded, or coated, with a drug that will inhibit or decrease cell proliferation and/or will reduce restenosis. In addition, the material may be treated to have active or passive surface components with drugs that will be advantageous for an extended time after the stent is exposed by bioresorption of the longitudinal structure.

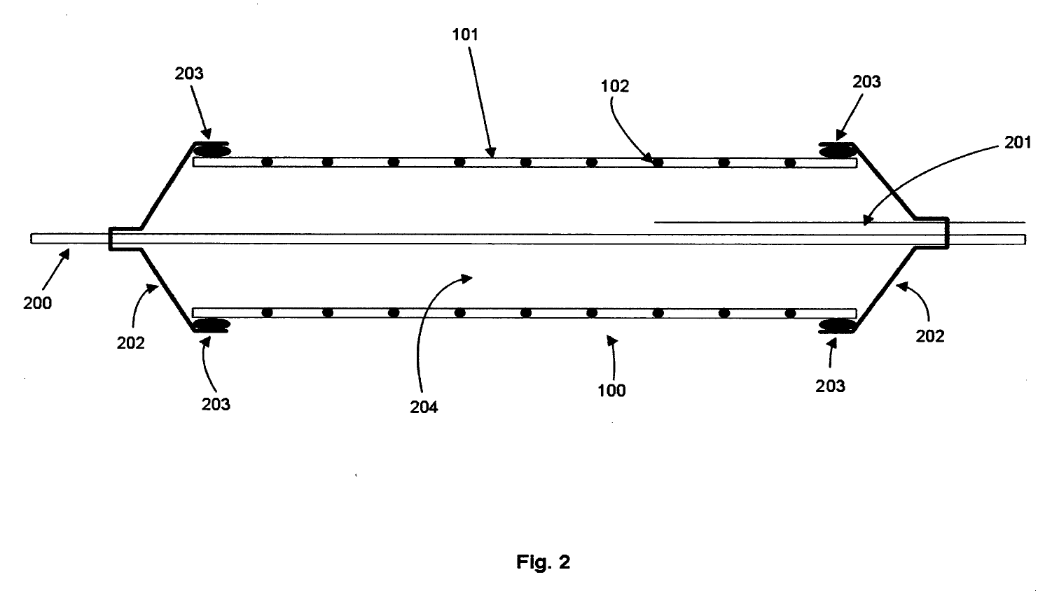

A covered stent delivery system according to the present invention will be understood with reference to FIG. 2. Stent 100, covered by stent cover 101, may be a component of a stent delivery system in accordance with the present invention by attaching, for example, sleeves 202 along each end of stent 100. Sleeves 202 may be adapted to be detachable from stent cover 101 after expansion of stent 100 within a vessel. As herein embodied, sleeves 202 may be constructed of the same material as stent cover 101. For purposes of this application, the term “cover” may include the material which is wrapped around stent 100 or encloses stent 100 (such that the stent may be considered embedded within the cover material) but in either event functions to facilitate balloon-type expansion of stent 100. It may also function to carry additional components (i.e., drugs) to the vessel wall once stent 100 is deployed. Stent cover 101 refers to the covering of stent 100 which remains implanted in the body vessel subsequent to deployment, but which may, if so desired, biodegrade over time.

Sleeves 202 are detachably secured to the longitudinally opposite ends of stent cover 101 along circumferential attachment rings or portions (indicated at 203). Sleeves 202 may be made of the same material as stent cover 101 or a different material. It will be understood that attachment rings 203 extend around the circumference of stent 100. Attachment rings 203 are not limited as shown in FIG. 2, as any surface portion of sleeves 202 can be attached to any surface portion of stent cover 101. The size of attachment rings 203 can be increased or decreased along the longitudinal axis of stent 100 depending upon the desired expansion characteristics of stent 100 during deployment. Attachment rings 203 may, for example, comprise a biocompatible adhesive or other suitable chemical or mechanical bond or attachment mechanism, such as an elastic-type pressure fit. Examples of suitable biocompatible adhesives, such as fluorinated thermoplastic polymer, are described in U.S. Pat. No. 6,168,619, the disclosure of which is hereby incorporated herein by reference.

Stent cover 101, sleeves 202 and attachment rings 203 are sufficiently impermeable to permit expansion of stent 100 to a desired diameter upon introduction of fluid through an inflation lumen 201 into a lumen 204. These components are capable of containing a sufficient amount of fluid within lumen 204, as the fluid is introduced through inflation lumen 201, to allow for an increase in fluid pressure sufficient to expand stent 100 to a desired diameter. It is to be understood that sleeves 202 remain attached to a catheter 200 throughout the duration of the stent deployment procedure and subsequent removal of catheter 200 from the living body. Alternatively, sleeves 202 can detach from catheter 200 during deployment, but it will be understood that sleeves 202 may subsequently be removed from the living body. As preferably embodied, upon substantially full expansion of stent 100 to its desired diameter, sleeves 202 will detach from stent cover 101 along attachment rings 203, allowing for removal of sleeves 202 with catheter 200. Stent 100 and stent cover 101 remain in their expanded state, maintaining patency of the body vessel. The pressure of the fluid within lumen 204 and the corresponding diameter of stent 100 at which sleeves 202 are automatically detached from stent cover 101 at attachment rings 203 can be regulated by adjusting the size of attachment rings 203 along the longitudinal axis of stent 100.

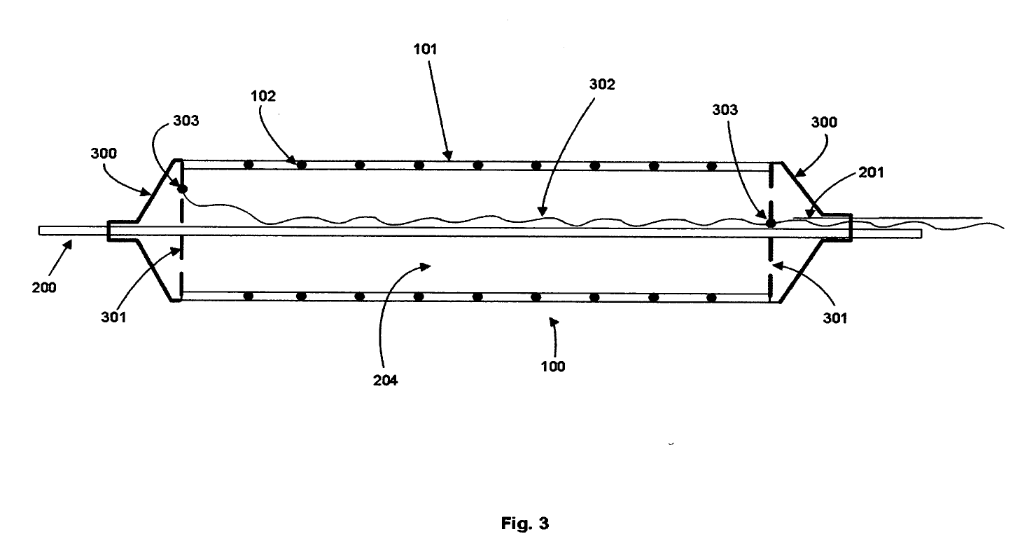

Another embodiment of a stent delivery system according to the present invention is illustrated in FIG. 3. Stent 100 is covered by, or embedded within, stent cover 101 and attached sleeves 300. Sleeves 300 are detachable from stent cover 101 along one or more separation line(s) 301 (alternatively referred to as frangible sections or webs). Separation line(s) 301 represents any separable connection which allows for separation of sleeves 300 from stent cover 101. The separable connection may be, for example, a tear-able connection. Alternatively, stent cover 101 and sleeves 300 may be integrally formed from continuous material wherein separation line 301 has a thinner cross-section which readily allows for separation. In a preferred embodiment, separation line 301 comprises an area of diminished material strength that allows for detachment of sleeves 300, such as, for example, perforations, scoring or frangible web. Separation line 301 can be formed after stent 100 has been encapsulated by cover 101, or preferably, during manufacture of cover 101 prior to encapsulation, such that separation line 301 is sufficiently impermeable to allow for expansion of stent 100 to a desired diameter upon the introduction of fluid into lumen 204. To facilitate the manual detachment of sleeves 300 from stent cover 101 along separation line 301, a separation wire 302, such as a catheter guide wire, or any other wire-type extension, may be provided. Separation wire 302 may extend proximally towards the operator and may terminate external to the patient's body, or be connected to an extension member that terminates external to the patient, such as a string, or other suitable extension means. Separation wire 302 may be attached to the cover at one or more location(s) 303 along separation line 301. It will be understood that location(s) 303 can be positioned anywhere along the circumferential extension of separation line 301. Furthermore, separation wire 302 may be attached to a plurality of location(s) 303 along separation line 301. As described herein, separation wire 302 may also be used to manually detach sleeves 202 from stent cover 101 at attachment area 203, as illustrated in FIG. 2.

In one embodiment of the invention, a pulling force exerted on the proximal end of separation wire 302 is transferred through separation wire 302 to location(s) 303 causing the detachment of sleeves 300 from stent cover 101 along separation line 301. Advantageously, the pulling force exerted on separation wire 302 may serve to tear or rip the perforations along separation line 301, resulting in detachment of sleeves 300 from stent cover 101. It will be understood that sleeves 300 remain attached to catheter 200 throughout the duration of the stent deployment procedure and subsequent removal of catheter 200 from the patient. Alternatively, sleeves 300 may detach from catheter 200, but are subsequently removed from the living body after deployment.

The separation means along separation line 301, such as, for example, perforations, scoring or frangible web, can be adapted to allow for automatic detachment of sleeves 300 from stent cover 101 after substantially full expansion of stent 100. This allows for elimination of separation wire 302. In that way, the separation means along separation line 301 may automatically separate by, for example, tearing or ripping, at a desired diameter of stent 100, thus detaching sleeves 300 from stent cover 101.

According to operation of the embodiment of the stent delivery system with reference to FIG. 3, after stent 100 has been positioned at a desired location within a body vessel, fluid is introduced into lumen 204 through inflation lumen 201. Stent cover 101, sleeves 300, and separation line 301 are, as explained above, sufficiently impermeable to allow for a build up of fluid pressure within lumen 204. The increase in pressure exerts an expansive force on stent 100, stent cover 101 and sleeves 300. Since stent cover 101 is attached to the structure of stent 100 (i.e., struts 102), this expansive force causes stent 100 to expand diametrically. When the desired diameter of stent 100 has been obtained, fluid is removed from lumen 204 through inflation lumen 201. A pulling force is exerted on the proximal end of separation wire 302 which is transferred to location(s) 303 detaching sleeves 300 from stent cover 101 along separation line 301. After detachment, stent 100 and stent cover 101 remain in an expanded state at the desired location within the body vessel. Sleeves 300 remain attached to catheter 200 after detachment from stent cover 101. Sleeves 300 and separation wire 302 are all removed from the body vessel with catheter 200 after deployment of stent 100 and stent cover 101. Alternatively, sleeves 300 may detach from catheter 200, but are subsequently removed from the living body after deployment.

The present invention provides an improved stent delivery system with a high degree of flexibility and deliverability, and a reduced system profile. The present invention also minimizes the tendency of a stent to “dog bone” during expansion. Further, the improved stent delivery system disclosed herein maximizes the efficacy of drug eluting stents by eliminating damage to the stent coating caused by interaction with an expanding balloon. The preceding detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention. This description will clearly enable one skilled in the art to make and use the invention, and describes multiple embodiments, adaptations, variations, alternatives and uses of the invention, including what is presently believed to be the best mode of carrying out the invention. Accordingly, it is not intended that the present invention be limited except by the appended claims.

The invention claimed is:

1. A stent delivery system comprising:
a stent having a substantially impermeable cover, the cover being detachably mounted to a catheter shaft, wherein the cover and the stent can be separated from the catheter shaft after substantially full expansion of the stent.

2. The stent delivery system according to claim 1, further including a coating on the cover.

3. The stent delivery system according to claim 2, wherein the coating is a drug eluting coating.

4. The stent delivery system according to claim 1, wherein a drug is embedded within the cover.

5. The stent delivery system according to claim 1, wherein the cover is biodegradable.

6. The stent delivery system according to claim 1, the cover and the catheter shaft further comprising:
a first sleeve attached to the catheter shaft, the first sleeve detachably mounted at a first end of the cover; and
a second sleeve attached to the catheter shaft, the second sleeve detachably mounted at a second end of the cover.

7. The stent delivery system according to claim 6, wherein said first and second sleeves are mounted to the cover by an adhesive.

8. The stent delivery system according to claim 6, wherein said first and second sleeves are mounted to the cover by an elastic member.

9. The stent delivery system according to claim 6, wherein said sleeves and said cover are made of essentially the same material which include a separation line for the first and second sleeves to detach from the cover.

10. The stent delivery system according to claim 9, wherein the separation line is formed from one of the group consisting of perforations, scoring and frangible web.

11. The stent delivery system according to claim 6, the first and second sleeves being automatically detachable from the cover upon substantially full expansion of the stent to a desired diameter.

12. The stent delivery system according to claim 6, the first and second sleeves being manually detachable from the cover after substantially full expansion of the stent to a desired diameter.

13. The stent delivery system according to claim 12, further including a separation wire that may be operated in such a way as to cause the first and second sleeves to separate from said cover.

14. The stent delivery system according to claim 13, further including an extension member attached to the separation wire.

15. The stent delivery system according to claim 14, wherein the extension member is a string which may be pulled to cause separation of said sleeves from said cover.

16. A method for deploying a stent within a body vessel, the method comprising the steps of:
(a) providing a stent delivery system comprising:
a stent having a substantially impermeable cover, the cover being detachably associated with a catheter shaft, wherein the cover and the stent can separate from the catheter shaft after substantially full expansion of the stent;
(b) inserting the stent delivery system;
(c) guiding the stent delivery system to a specific location;
(d) introducing fluid through an inflation lumen;
(e) expanding the stent and the cover;
(f) providing for the stent and the cover to become separated from the catheter shaft; and
(g) removing the catheter shaft and the inflation lumen.

17. The method according to claim 16, further including providing a coating on the cover.

18. The method according to claim 17, wherein the coating is a drug eluting coating.

19. The method according to claim 16, wherein a drug is embedded within the cover.

20. The method according to claim 16, wherein the cover is biodegradable.

21. The method according to claim 16, further comprising:
securing a first sleeve to the catheter shaft, and detachably mounting said first sleeve to a first end of the cover; and
securing a second sleeve to the catheter shaft, and detachably mounting said second sleeve to a second end of the cover.

22. The method according to claim 21, wherein said first and second sleeves are mounted to the cover by an adhesive.

23. The method according to claim 21, wherein said first and second sleeves are mounted to the cover by an elastic member.

24. The method according to claim 21, wherein said sleeves and said cover are made of essentially the same material which include a separation line for the first and second sleeves to detach from the cover.

25. The method according to claim 24, wherein the separation line is formed from one of the group consisting of perforations, scoring and frangible web.

26. The method according to claim 21, the first and second sleeves being automatically detachable from the cover upon substantially full expansion of the stent to a desired diameter.

27. The method according to claim 21, the first and second sleeves being manually detachable from the cover after substantially full expansion of the stent to a desired diameter.

28. The method according to claim 27, further including a separation wire that may be operated in such a way as to cause the first and second sleeves to separate from said cover.

29. The method according to claim 28, further including an extension member attached to the separation wire.

30. The method according to claim 29, wherein the extension member is a string which may be pulled to cause separation of said sleeves from said cover.
